(19) United States
(12) Patent Application Publication
(10) Pub. No.: US 2024/0245444 A1
(43) Pub. Date: Jul. 25, 2024

(54) SURGICAL SYSTEM AND METHOD FOR OPERATION THEREOF

(71) Applicant: Erbe Elektromedizin GmbH, Tuebingen (DE)

(72) Inventors: Peter SELIG, Nehren (DE); Sebastian SCHLEEHAUF, Tuebingen (DE); Yannick DANIEL, Balingen (DE)

(21) Appl. No.: 18/408,910

(22) Filed: Jan. 10, 2024

(30) Foreign Application Priority Data
Jan. 25, 2023 (EP) 23153318.3

(51) Int. Cl. A61B 18/12

(52) U.S. Cl. CPC A61B 18/12; A61B 2018/00827

(57) ABSTRACT

Surgical systems as well as methods for the operation thereof having a surgical instrument connected to a surgical apparatus and controlled by the latter. The surgical apparatus controls at least one operation parameter (B) of the surgical instrument. For example, the operation parameter (B) can be a parameter of an electrical output signal, such as an amplitude and/or a frequency and/or a wave form. The surgical system has an evaluation unit that is configured to identify the currently carried out or planned type of use (V) of the surgical instrument or the entire surgical system, wherein the type of use (V) is characteristic for the type of surgery that is to be carried out. At least one pattern parameter (M) is assigned to the identified type of use (V), which can be compared to a respectively assigned operation parameter (B). Based on the result of the comparison, at least one measure (X) can be started, e.g. the output of an information (INF) and/or the adaption of at least one operation parameter (B).

This application claims priority to European Patent Application No. 23153318.3, filed Jan. 25, 2023, the entirety of which is incorporated by reference herein

The invention refers to a surgical system comprising a surgical instrument and a surgical apparatus configured for connection of a surgical instrument. The surgical instrument is particularly an electrosurgical instrument having at least one electrode. Alternatively or additionally, the surgical instrument can also be an ultrasound instrument or a water jet surgical instrument. The surgical instrument can be operated exclusively electrically and for this purpose can be supplied with an electrical output power by the surgical apparatus. In addition or as an alternative, at least one fluid can be provided by the surgical apparatus for operation of the surgical instrument, e.g. a gas for creation of a plasma, such as argon or another inert gas, and/or a liquid, such as water for the water jet surgery.

In the prior art different approaches have been prosecuted in order to improve and optimize the operation of such surgical systems.

US 2018/0028088 A1 proposes to use different monitoring data from an operation room in order to monitor the progress of a surgery and to potentially indicate surgery risks. For this purpose, different monitoring sensors are used in the operation room, e.g. electroencephalography sensors (EEG sensors) by which the surgery team is equipped or monitoring cameras in the operation room. The data of the monitoring sensors can be evaluated by statistical methods.

A monitoring method of a surgery is known from WO 2022/157695 A1. In this method a surgical system is used in which operating data of an instrument and sensor data of an operating person using the instrument are submitted to an evaluation unit. The actions of the operating person are evaluated and feedback to the operating person is created, who can adapt the use of the instrument thereupon. For example, movement or biomarkers can be arranged on the operating person that can be detected by a respective detection system.

EP 3 895 176 A1 discloses a method and a system for extraction of a actual duration of a surgical operation. Thereby the sum of the time durations is considered as actual surgical operation during which a patient is treated by a surgical tool. In order to determine this duration, the total duration of a surgical method is determined and therefrom time durations are subtracted during which no surgical treatment takes place.

In addition to monitoring a surgical progress, it is also known to monitor at least one operation parameter of a surgical system and to adapt a setting of the surgical system based thereupon. For example, EP 3 876 238 A1 describes such an approach. In doing so, modes particularly provided by the surgical system for operation of the connected surgical instrument shall be adapted, discarded or re-created.

A system for improvement of the efficiency of electrosurgical generators is apparent from EP 3 167 834 A1. The generator comprises an inverter for producing a HF-signal for the connected electrosurgical instrument. The power efficiency of the inverter is determined. The control of the inverter is complemented by a number of switch-off periods, if the power efficiency of the inverter reaches a threshold value. In doing so, the efficiency of the generator shall be improved at low output power levels.

In DE 10 2009 024 612 A1 a surgical system is described having a surgical apparatus for providing an HF-output voltage as well as an electrosurgical instrument connected thereto. In order to adjust operation parameters for the electrosurgical instrument sufficiently accurately, the electrical load varying dependent on the treated tissue is determined. Depending on this load, a haptically perceivable signal is created on the surgical instrument for the operating person. The operating person can thereupon adapt the use of the surgical instrument, such as the cutting speed and/or the cutting depth.

The purpose of the present invention is to further improve the use of a surgical system. It can be considered as object of the present invention to optimize the use of the surgical system during a current use and/or for future uses.

The surgical system according to the present invention comprises a surgical apparatus and a surgical instrument connected thereto. The surgical instrument is particularly an electrosurgical instrument. The surgical apparatus is configured to supply the connected surgical instrument with the required electrical power and/or the required operating media. Electrical power or energy can be provided as high frequency (HF) output signal. Depending on the configuration of the surgical instrument, additionally or alternatively to the electrical energy, operating media can be provided, such as a gas or a liquid. For example, the gas can be required and used for plasma creation at the surgical instrument. For supply of the surgical instrument with electrical power and/or an operating medium, the surgical apparatus can comprise a supply unit.

The surgical apparatus comprises in addition a control unit. For example, the control unit can be configured to directly control the supply unit and/or the surgical instrument. The control unit can set at least one operation parameter of the surgical instrument.

The surgical system comprises in addition an evaluation unit. The evaluation unit can be, at least in part, a component of a surgical apparatus. The evaluation unit can also be partly or completely realized externally from the surgical apparatus. In any case, the evaluation unit is communicatively connected to the surgical apparatus.

The evaluation unit is configured to identify a currently executed type of application or type of use of the surgical instruments or an upcoming planned type of application or type of use of the surgical instrument. The type of application or type of use of the surgical instrument particularly means the type of surgical operation, which is carried out by the surgical system or the surgical instrument, i.e. the type of surgical treatment. The type of application, particularly the type of surgical operation or surgical treatment is characterized by a tissue type or multiple tissue types that are treated during the surgical operation, e.g. cut, coagulated, fusioned, etc. Thereby the type of application of the surgical system can be characterized by a sequence or temporal progress of the influence on different tissue types. The types of application or types of use of the surgical instrument can distinguish from one another by the surgical field (gastroenterology, pneumology, gynecology, urology, abdominal surgery, etc.) to which it is associated and/or by the at least one treated tissue type. Examples for types of application or types of use of the surgical instrument can be: biopsies or myotomies or ectomies respectively on different tissue types (skin and/or muscle tissue and/or fat tissue and/or internal organs, etc.), bronchial recanalization, etc.

The identification of a currently executed or planned type of application can be based on the detection of at least one operation parameter during a current use of the surgical instrument. For example, types of application can be distinguished from one another in that at least one operation parameter is evaluated. For example, a distinction can be made possible by one or more of the following evaluations:
occurrence of different settings of an operation parameter or multiple operation parameters;
order of occurrence of different settings of an operation parameter or multiple operation parameters;
time duration of one or more settings of an operation parameter or multiple operation parameters;
temporal progress of an operation parameter or multiple operation parameters;
a combination of settings of multiple operation parameters to a respective observation point in time.

For identification of the type of use of the surgical system the at least one operation parameter can be determined and can be compared with an assigned pattern parameter of one or more usage patterns. If a sufficient similarity between the at least one operation parameter of the current application and the at least one pattern parameter of a usage pattern is determined, it can be concluded therefrom that the current type of use of the surgical instrument coincides with the type of use of the usage pattern. In other words, the current type of use corresponds to the known type of use of the sufficiently similar usage pattern. In this manner it is possible to identify the type of use of the surgical instrument currently being performed. In this type of identification it is not necessary to separately submit the type of use to the surgical system, e.g. by an input via a user interface of the surgical system or by transmission of the type of application by a data management system to which the surgical system can be connected.

For checking the similarity known methods can be used, as they are also used in the field of pattern recognition. A sufficient similarity is determined, if the at least one operation parameter is identical to the respectively assigned pattern parameter and/or if the differences are within a tolerance range.

Additionally or alternatively, it is also possible to define the planned type of application for an upcoming surgical operation. For this purpose, the operating person can input the planned type of application, particularly the type of surgical treatment to be carried out, e.g. by an operating interface of the surgical system, particularly an operating interface of the surgical apparatus. The type of use can also be stored in a data management system (e.g. database and/or data processing system), particularly a hospital information system (HIS). The surgical system can be communicatively connected with the data management system by a communication network and can retrieve the planned type of use from the data management system or it can be submitted automatically by the data management system. The data management system can be communicatively connected with a surgical system and particularly the surgical apparatus in a wireless and/or wired manner. The surgical apparatus can comprise for this purpose a respective communication interface.

If the evaluation unit of the surgical apparatus determines the identification of the type of use based on the current application of the surgical instrument, the identification is carried out at least slightly delayed in time after the start of use of the surgical instrument. Regularly the identification of the type of use based on the at least one operation parameter and the pattern comparison with the at least one usage pattern can be carried out sufficiently early during the progress of a surgical operation, so that the type of use is at least known for a portion and particularly a prevailing portion of the entire surgical operation, which has still to be carried out.

After the type of use has been identified, the at least one operation parameter of the surgical instrument can be compared with the at least one pattern parameter of a usage pattern. The usage pattern is assigned to the identical type of use as the planned or current application of the surgical system. For example, the usage pattern can be a common or ideal use of the identified type of application. Depending on a result of the comparison, a measure to be carried out can be initiated or carried out by the evaluation unit. For example, a deviation between one or more operation parameters from the respectively assigned pattern parameter can be indicated, e.g. quantitatively and/or qualitatively. Based thereupon also an evaluation of the current use can be carried out as an option.

The at least one measure that can be initiated or carried out by the surgical apparatus and particularly the evaluation unit can be, for example, the output of information to an operating person or a member of the surgery team. The information can be output as optical information on a screen and/or as acoustic information via a speaker and/or as haptical information on the surgical instrument. The information can comprise an information or multiple of the information indicated in the following in any combination:
a warning, if a deviation of the at least one operation parameter from the respectively assigned pattern parameter is present;
a recommendation for a modified setting of the operation parameter or of at least one of the operation parameters;
information concerning one or more upcoming steps that are necessary in the context of the surgical operation.

The at least one measure can additionally or alternatively comprise to automatically modify one of the operation parameters or multiple of the operation parameters or to indicate a proposal for an adaption of one or more of the operation parameters.

Based on the result of a comparison, information can be output to the operating person and/or a member of the surgery team and/or a planning instance, as at least one measure. Such an information can comprise one or more of the following aspects, for example:
a determined remaining time duration till the termination of the application or the surgical operation;
a determined time duration for at least one subsequent application step of the application or the surgical operation;
a resource (material and/or person) required for at least one subsequent application step.
   This information can be determined and output, if the comparison shows a deviation between the at least one operation parameter and the at least one pattern parameter or also if such a deviation has not been determined. Based on this information, for example, the occupation of an operation room, cleaning and sterilization processes, medication or instrument requirements, personnel deployment or any other resource can be planned and/or adapted.

The determination and initiation or execution of at least one suitable measure is based on the identification of the current or planned type of application according to the present invention, i.e. particularly on the currently executed or planned type of treatment using the surgical system. The at least one measure can, therefore, be very precisely adapted to the actual situation and particularly the current surgical treatment, which in turn is a remarkable added value of the surgical system. For example, the danger of wrong settings of operation parameters can be reduced or completely eliminated. Undesired impairments of the tissue during treatment can be reduced or avoided.

The at least one measure can also comprise the automatic adaption of the operation parameter or of one of the operation parameters.

The at least one operation parameter can be an electrical output signal and/or an optical output signal and/or a fluid parameter and/or a power parameter that is provided by the surgical apparatus to the surgical instrument. For example, one or more of the following parameters can be used as operation parameters:
an amplitude and/or a frequency of an electrical output signal, particularly a high frequency output signal, e.g. an output voltage and/or an output current;
a crest factor of the electrical output signal;
a duty cycle of the electrical output signal, if the electrical output signal is a pulse width modulated electrical output signal;
a wave form of the electrical output signal or a section (e.g. a half wave) of the electrical output signal;
a mode of the electrical output signal;
a fluid parameter of a fluid provided to the surgical instrument, e.g. a fluid pressure and/or a volume flow rate;
an optical output signal.

The electrical output signal can be an electrical output voltage and/or an electrical output current. The operation parameter can be set optionally only for the output voltage or only for the output current or for the output voltage as well as for the output current.

In an embodiment the evaluation unit is in addition configured to determine a deviation between the operation parameter or at least one of the used operation parameters during the current application of the surgical instrument compared with the respectively assigned pattern parameter as the result or as one of the results of the comparison. The at least one measure is particularly determined and initiated depending on the deviation. It is particularly advantageous, if the measure or one of the measures is determined and initiated only if the deviation fulfills a deviation criterion. The deviation criterion can be a quantitative or qualitative deviation criterion. For example, it is possible to initiate a specific measure only if the determined deviation exceeds a threshold defined by the deviation criterion.

During use of the surgical instrument the operation parameter or one of the adjustable operation parameters can be determined and stored continuously or in a time-discrete manner at predefined monitoring points in time, for example. In this manner a temporal sequence of the settings of one or more operation parameters and/or a continuous temporal progress of the operation parameter can be determined and stored optionally.

Preferably the surgical apparatus is configured to store the operation parameter or at least one of the operation parameters during use of the surgical instrument and/or to transmit the operation parameter or at least one of the operation parameters during use of the surgical instrument to the data management system if the surgical system is communicatively connected to the data management system. The communication connection can also be established temporarily, so that buffered operation parameters can be transmitted during the established communication connection. In addition or as an alternative, the surgical system and particularly the surgical apparatus can comprise an interface (e.g. USB-interface) to which a storage medium can be connected in order to transmit stored data of the at least one operation parameter to an external storage medium.

Advantageous embodiments of the invention are derived from the dependent claims, the description and the drawing. In the following preferred embodiments of the invention are explained in detail based on the drawing. The drawing shows:

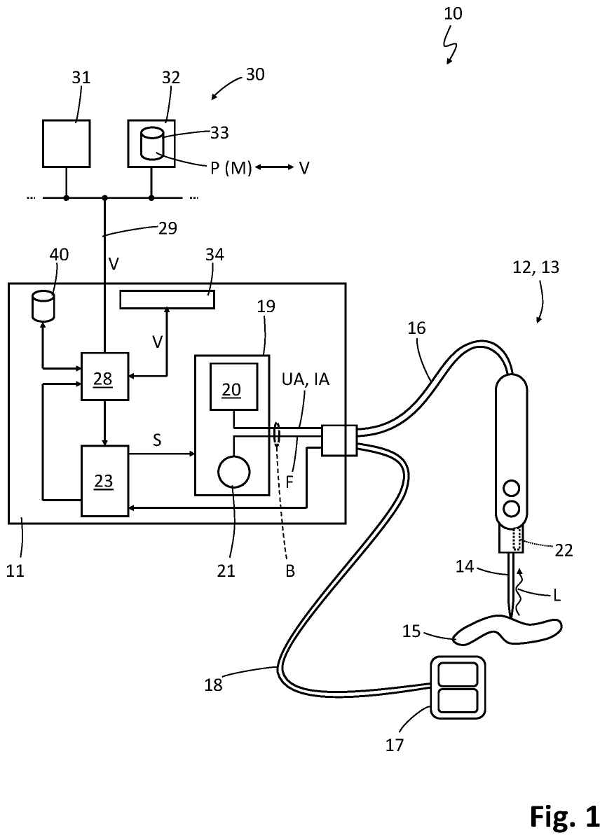

FIG. 1 a schematic illustration of an embodiment of a surgical system,

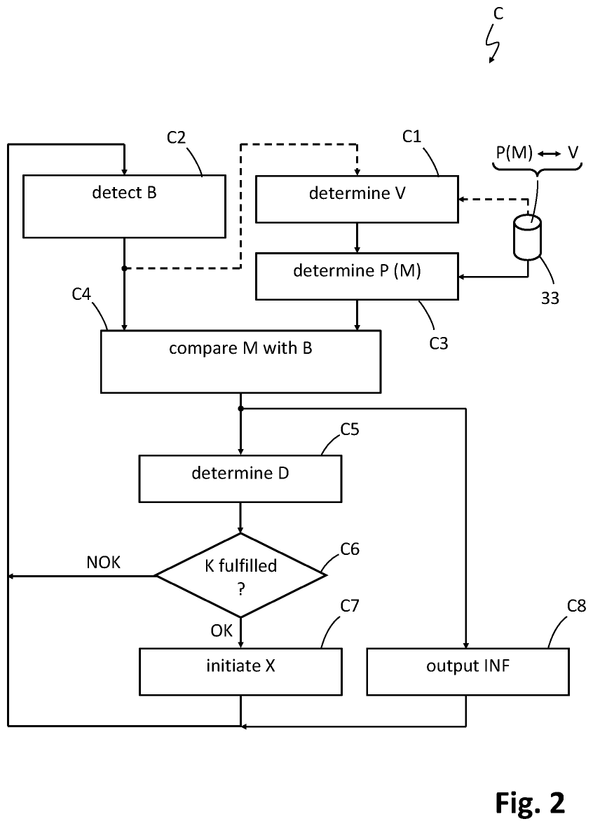

FIG. 2 a flow diagram of an embodiment of a method for operating a surgical system, particularly the surgical system of FIG. 1,

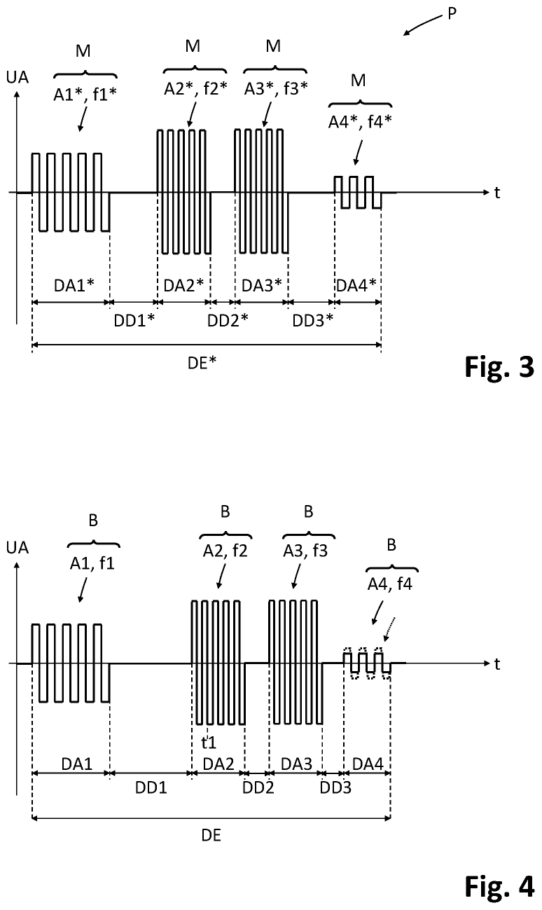

FIG. 3 an exemplary schematic illustration of multiple pattern parameters of a pattern use of an electrosurgical instrument of a surgical system and

FIG. 4 an exemplary schematic illustration of at least one operation parameter during use of an electrosurgical instrument dependent on the point in time.

In FIG. 1 a surgical system 10 is illustrated schematically in the type of a block diagram comprising a surgical apparatus 11 as well as a surgical instrument 12 connected to the surgical apparatus 11. In the embodiment the surgical instrument 12 is an electrosurgical instrument 13. The electrosurgical instrument 13 has at least one electrode 14. By the at least one electrode 14, the electrosurgical instrument 13 can influence tissue 15 of a patient.

The surgical instrument 12 is connected to the surgical apparatus 11 via an instrument line 16. The instrument line 16 can provide an electrical and/or fluidical and/or optical connection between the surgical instrument 12 and the surgical apparatus 11. In case of an electrosurgical instrument 13, at least an electrical connection is established.

In the embodiment illustrated in FIG. 1 the electrosurgical instrument 13 is configured as monopolar instrument. If due to the treatment with the electrosurgical instrument 13 a current flows into the tissue 15, a neutral electrode 17 on the patient provides for establishment of a closed treatment current circuit. The neutral electrode 17 is electrically conductively connected to the patient and in addition electrically connected to the surgical apparatus 11 via a neutral electrode line 18. During use of a bipolar electrosurgical instrument 13 neutral electrode 17 can be omitted.

The surgical apparatus 11 is configured to supply electrical power or energy and/or at least one operation medium to the surgical instrument 12. For the supply, surgical apparatus 11 comprises a supply unit 19. By the supply unit 19 an electrical output signal and/or a fluid and/or an optical signal and/or energy in form of laser light, etc. is provided to the surgical instrument 12. In the embodiment illustrated in FIG. 1 the supply unit 19 comprises a generator 20 in order to apply an electrical output signal to the at least one electrode 14 of the electrosurgical instrument 13, which can be provided as an electrical output voltage UA and/or an electrical output current IA. The electrical output voltage UA and/or the electrical output current IA can be particularly a high frequency electrical output signal in a frequency range from, e.g. 100 kHz or 300 kHz to 4 MHZ, for example. Optionally the supply unit 19 can comprise also a gas source 21 in addition to generator 20 in order to provide a gas for a plasma creation and/or a purge gas to the electrosurgical instrument 13.

The surgical instrument 12 can comprise in addition at least one sensor 22 in order to create a measurement signal during the application of the surgical instrument 12 that describes a parameter to be measured and to transmit it via the instrument line 16 to the surgical apparatus 11 and particularly a control unit 23 of the surgical apparatus 11. For example, light L, which is created during influence on the tissue 15 by the at least one electrode 14, can be detected by sensor 22. For example, light L can be emitted due to a spark formation between the at least one electrode 14 and the tissue 15. The measurement signal can be analyzed in the control unit 23. For example, a spectral analysis of light L can be carried out. From the measurement signal at least one operation parameter B can be determined. Based on the measurement signal and/or an operation parameter derived therefrom, the operation of the surgical instrument 12 can thus be monitored and if required operation parameter B or one of the operation parameters B can be controlled in a closed control loop.

In general, control unit 23 is configured for control of the at least one operation parameter B by which the surgical instrument 12 is operated. This operation parameter B can be set by surgical apparatus 11. For this purpose, control unit 23 can transmit a control signal S to the supply unit 19, for example, in order to set or adjust one or more operation parameters B.

The surgical apparatus 11 has, in addition, an evaluation unit 28. The evaluation unit 28 is part of the surgical apparatus 11 according to the example, but can alternatively to FIG. 1 also be arranged external from surgical apparatus 11 and can be communicatively connected to surgical apparatus 11, e.g. by a communication network 29. The connection via communication network 29 can be wired or wireless.

The evaluation unit 28 and the control unit 23 are communicatively connected according to the example or can alternatively to the illustration in FIG. 1 also be realized as common unit or assembly. In this manner operation parameter B adjusted and/or set by control unit 23 can be transmitted to the evaluation unit 28. In addition, the evaluation unit 28 can be connected with sensor 22 indirectly via control unit 23 or alternatively also directly, so that also the measurement signal provided by sensor 22 can be made available for the evaluation unit 28.

In the embodiment of the surgical system 10 illustrated in FIG. 1, evaluation unit 28 is communicatively connected via communication network 29 with a data management system 30. The data management system 30 can comprise one or more external computing units 31 and/or databases 32. In the embodiment the data management system 30 comprises a database 32 and a database memory 33. In the database memory 33 multiple usage patterns P are stored. At least one pattern parameter M is assigned to each usage pattern P. At least one of the pattern parameters M, multiple of the pattern parameters M or all of the pattern parameters M respectively correspond to one assigned operation parameter B. Each usage pattern P is defined by the value of the associated pattern parameter M, wherein the value of each pattern parameter M can be constant or can vary in a time-dependent manner. For example, each pattern parameter M can be a temporal sequence of different values in directly or indirectly following time intervals.

The data management system 30 can be a hospital information system (HIS), for example. In addition to the database memory 33 comprising the usage pattern P, also additional database memories can be present, in which surgery plans or other resource plans, patient data, etc. can be stored, for example. The surgical system 10 can optionally access such additional data and information via the communication network 29, which is provided by the data management system 30 (e.g. hospital information system).

In a modified embodiment the data management system 30 and/or the at least one external computing unit 31 and/or the at least one database 32 can also be integrated into the surgical apparatus 11 or can be arranged inside surgical apparatus 11 (not illustrated in FIG. 1).

As operation parameter B all of the settings can be used that characterize the operation of the surgical instrument 12, particularly such parameters that are set by surgical apparatus 11 and can be modified or adjusted optionally prior to and/or during an application. In the embodiment illustrated here one or more of the following operation parameters B can be used as the at least one operation parameter B:
the amplitude of the output voltage UA;
the frequency of the output voltage UA;
the wave form of the output voltage UA;
a crest factor of the output voltage UA;
the amplitude of the output current IA;
the frequency of the output current IA;
the wave form of the output current IA;
a crest factor of the output current IA;
a tissue impedance of the tissue 15;
an impedance of the treatment current circuit from the surgical apparatus 11 to the surgical instrument 12 and via the tissue 15 (and as an option a neutral electrode 17) back to the surgical apparatus 11;
a fluid pressure of a fluid provided by the fluid source 21;
a volume flow rate of the fluid provided by the fluid source 21.

In the embodiment surgical apparatus 11 has a user interface 34. Via the user interface an operating person can carry out inputs and can, for example, set the at least one operation parameter B. For this purpose, user interface 34 is communicatively connected to the control unit 23. The control unit 23 can inform the operating person via the user interface 34 about current operation conditions and/or can output other information. The user interface 34 can comprise a touch screen for input and output of data and information, for example. In addition or as an alternative, also other devices such as a speaker, microphone, keys, buttons, etc. can be part of the user interface 34. In the embodiment the evaluation unit 28 is communicatively connected to user interface 34 directly or indirectly via control unit 23.

Via the user interface 34 or the data management system 30, information can be submitted to the surgical system 10 that describes a planned type of use V of the surgical instrument 12. The type of use V characterizes the use of the surgical instrument 12 during treatment or during influence on tissue 15 of a patient. The type of use V of the surgical instrument 12 is particularly characterized by at least one of the parameters indicated in the following:
a total duration DE of the surgical instrument from a starting point in time ts to a termination point in time te of a surgical operation;
a combination or sequence of different settings of an operation parameter B or a group of multiple or all operation parameters B;
a continuous temporal progress of an operation parameter B or a group of multiple or all operation parameters B;
at least one activation duration DA during the total duration DE in which the surgical instrument 12 is activated and influences the tissue 15 of the patient;
at least one deactivation duration DD during the total duration DE during which the surgical instrument 12 is deactivated and is not used for influencing tissue 15 of the patient, wherein a deactivation duration DD can separate two subsequent activation durations DA from one another;
a parameter derived from an operation parameter B, e.g. an impedance of the tissue 15, which can be determined based on the output voltage UA and the output current IA, for example.

In FIG. 4 a temporal progress for the output voltage UA during a surgical treatment is exemplarily illustrated. This illustration is only schematic and exemplary for explanation of the invention principle and can vary dependent on the type of use V of the surgical instrument 12.

The surgical operation starts with the starting point in time ts and ends with the termination point in time te. During the total duration DE by way of example for activation durations DA, namely a first activation duration DA1, a second activation duration DA2, a third activation duration DA3 and a fourth activation duration DA4 are provided. Between two subsequent activation durations one deactivation duration DD is respectively present, in the embodiment a first deactivation duration DD1, a second deactivation duration DD2 and a third deactivation duration DD3, during which the surgical instrument 12 (in the embodiment electrosurgical instrument 13) is not used for influencing tissue 15.

During each activation duration DA an operation parameter B or a set of multiple operation parameters B of the surgical instrument 12 are set. Only exemplarily here the amplitude A of the output voltage UA and the frequency f of the output voltage UA are illustrated by way of example. In the embodiment the setting of the output voltage UA is as follows: a first amplitude A1 and a first frequency f1 during the first activation duration DA1, a second amplitude A2 and a second frequency f2 during the second activation duration DA2, a third amplitude A3 and a third frequency f3 during the third activation duration DA3 and a fourth amplitude A4 and a fourth frequency f4 during the fourth activation duration DA4. In the example the second amplitude A2 and the third amplitude A3 are equal and in addition the second frequency f2 and the third frequency f3 can be equal. The amplitude and frequencies during the activation durations DA1 to DA4 can be respectively equal or can have different values, which depends on the type of use V.

Additionally or alternatively to the output voltage UA, one or more other operation parameters B can be set during the different activation durations DA1 to DA4 in a constant or time-varying manner. For example, in addition the wave form of the output voltage UA during one or more activation durations can deviate from the exemplarily illustrated square wave progress.

The temporal sequence or the temporal progress of the operation parameters B with which the surgical instrument 12 is used during surgical operation characterizes the type of use Vi (i=1, 2, 3, . . . , n) among multiple possible types of use V1, V2, V3, . . . , Vn. For example, one type of use can be assigned respectively to different treatments (surgical operation on an inner organ or on skin tissue or on muscle tissue). For different surgical operations or treatments of a patient different types of use V are therefore provided, which are particularly characterized by the setting of the operation parameters B or their respective temporal progress.

If at the starting point in time ts of the use of the surgical instrument 12 it is unknown which type of use V is carried out currently, the type of use V can also be recognized from the settings and particularly the temporal progress of one operation parameter B or multiple operation parameters B. For this purpose, for example, the temporal progresses of one or multiple operation parameters B of the type of use V, which is carried out currently (FIG. 4) can be compared to respective pattern parameters M of one or more usage patterns P. If a sufficient similarity with the used pattern P exists, it can be determined that the current use is a type of use V, which is described by the identical or sufficiently usage pattern P. For this purpose, known methods (e.g. pattern recognition) can be used. As soon as the temporal progress of the at least one operation parameter B of the currently carried out application of the surgical instrument 12 allows a distinction between the available usage patterns P, it can be determined that the type of use of the coinciding usage pattern P corresponds to the current type of use. For example, this can occur at a first point in time t1 (FIG. 4) at which a sufficient similarity with the usage pattern P of FIG. 3 has been determined and a sufficient distinction compared to other usage patterns exist.

During the check of similarity between operation parameters B of a current use and the respectively assigned pattern parameter M individual parameters can be given more weight than others. For example, the activation durations DA1 to DA4 and/or operation parameters B changing during one of the activation durations DA1 to DA4 and/or operation parameters B, distinguishing at least between two of the activation durations DA1 to DA4, can be considered with higher significance during the similarity check than deactivation durations DD1 to DD3. For the example of the use illustrated in FIG. 4, a usage pattern P is illustrated in FIG. 3 schematically only by way of example that characterizes a specific type of use V and comprises typical settings or temporal progresses of one or more pattern parameters M—here, for example
the pattern amplitude A1*-A4* of the output voltage UA,
the pattern frequency f1*-f4* of the output voltage UA,
the pattern activation duration DA1*-DA4*,
the pattern deactivation durations DD1*-DD3*
the pattern total duration DE* of the treatment.

As already explained, it is also possible to set the planned upcoming type of use V so that a determination during the application can be omitted and the type of use V of the surgical instrument 12 is already known at the starting point in time ts.

With reference to FIG. 2 an example of a method C for operating or using the surgical system 10 is explained.

In a first method step C1 the type of use V is identified with which the surgical apparatus 11 shall be used during an upcoming planned surgical operation. For example, the planned type of use V can be preset via the user interface 34 or the data management system 30.

Beginning with the starting point in time ts, the operation parameters B or at least one of the operation parameters B, multiple operation parameters B or all operation parameters B are detected that the surgical system 10 sets for the operation of the surgical instrument 12. The second method step C2 can—as explained above—also be carried out temporally prior to the first method step C1 in a modification of the method illustrated in FIG. 2, in order to check similarity of the type of use V based on a pattern comparison with usage patterns P available in the database memory 33.

After the type of use V of the surgical instrument 12 has been identified, the usage pattern P assigned to this type of use V can be determined by accessing the database memory 33 (third method step C3).

For example, the usage pattern P can describe a typical or ideal progress of a surgical operation between the starting point in time ts and the termination point in time te with the respectively associated settings of the at least one operation parameter B. The pattern parameters M defined by the usage pattern P are comparison values or setpoint values for the operation parameters B, which are provided to the surgical instrument 12 by surgical apparatus 11 during a surgical operation.

In a fourth method step C4 then the at least one operation parameter B determined in the second method step C2 can be compared with the respectively assigned pattern parameter M of the usage pattern P. Between one or more of the available pairs of one operation parameter B and one assigned pattern parameter M respectively, a deviation D can then be determined (fifth method step C5).

Subsequent to the fifth method step C5 the determined deviation D or each determined deviation D can be evaluated whether an assigned deviation criterion K is fulfilled (sixth method step C6). If the deviation criterion K is not fulfilled (branch “NOK” out of sixth method step C6), the method C can be again continued with the detection of the at least one operation parameter B in the second method step C2.

If however the deviation criterion K is fulfilled (branch “OK” out of sixth method step C6), the method C is continued in a seventh method step C7. In the seventh method step C7 a suitable measure X is initiated or carried out by evaluation unit 28 of the surgical apparatus 11. Thereby arbitrary different measures X are possible, such as the output of a warning or information via the user interface 34 to an operating person and/or the automatic adaption of at least one operation parameter B in order to reduce the respective deviation D to the respectively assigned pattern parameter M.

For example, one or more of the following measures X can be initiated or carried out:
output of a warning or information about the deviation D;
output of a recommendation about the adaption of one or more adjustable operation parameters B;
automatic change of one or more of the adjustable operation parameters B.

Additionally or alternatively to the method steps C5-C7 following the fourth method step C4, a measure can be initiated or carried out in an eighth method step C8 in which one or more pieces of information INF are output, e.g. via user interface 34. The measure defined in eighth method step C8 can be independent from a determined deviation D. It can optionally also be output under consideration of the deviation D as measure in the seventh method step C7. The information INF can comprise one or more of the following pieces of information:
an information referring to one or more upcoming steps of the surgical operation during the further progress of the surgical operation up to the termination point in time te;
an information referring to one or more required resources for one or more steps of the surgical operation to be carried out;
an information referring to the time duration for one, multiple or all steps of the surgical operation that still have to be carried out;
the transmission of the expected termination point in time te of the surgical operation to the data management system 30;
the request of one or more resources via the data management system 30.

In another embodiment of the invention the entire progress of a surgical operation from a starting point in time ts up to a termination point in time te can be detected and stored, e.g. in an optionally provided internal memory 40 of the surgical apparatus 11 and/or in the data management system 30. This can be carried out for the purpose of documentation, for the purpose of assessment or further education or training of surgery personnel.

The surgical system 10 has been explained above by way of example based on an electrosurgical system having an electrosurgical instrument 13. In modified embodiments the surgical system 10 can also have a water jet surgical instrument or an ultrasound surgical instrument or another surgical instrument 12. It is also possible to operate and use multiple different surgical instruments 12 during a surgical operation.

The invention refers to a surgical system 10 as well as a method C for the operation thereof. The surgical system 10 has a surgical instrument 12, e.g. an electrosurgical instrument 13, which is connected to a surgical apparatus 11 and is controlled by the latter. The surgical apparatus 11 controls at least one operation parameter B of surgical instrument 12. For example, the operation parameter B can be a parameter of an electrical output signal, such as an amplitude and/or a frequency and/or a wave form. The surgical system 10 has an evaluation unit 28 that is configured to identify the currently carried out or planned type of use V of the surgical instrument 12 or the entire surgical system 10, wherein the type of use V is characteristic for the type of surgery that is to be carried out. At least one pattern parameter M is assigned to the identified type of use V, which can be compared to a respectively assigned operation parameter B. Based on the result of the comparison, at least one measure X can be started, e.g. the output of information INF and/or the adaption of at least one operation parameter B.

### LIST OF REFERENCE SIGNS

10 surgical system
11 surgical apparatus
12 surgical instrument
13 electrosurgical instrument
14 electrode
15 tissue
16 instrument line
17 neutral electrode
18 neutral electrode line
19 supply unit
20 generator
21 fluid source
22 sensor
23 control unit
28 evaluation unit
29 communication network
30 data management system
31 computing unit
32 database
33 database memory
34 user interface
40 internal memory
A1 first amplitude
A1* first pattern amplitude
A2 second amplitude
A2* second pattern amplitude
A3 third amplitude
A3* third pattern amplitude
A4 fourth amplitude
A4* fourth pattern amplitude
B operation parameter
C method
C1 first method step
C2 second method step
C3 third method step
C4 fourth method step
C5 fifth method step
C6 sixth method step
C7 seventh method step
C8 eighth method step
D deviation
DA activation duration
DA1 first activation duration
DA1* first pattern activation duration
DA2 second activation duration
DA2* second pattern activation duration
DA3 third activation duration
DA3* third pattern activation duration
DA4 fourth activation duration
DA4* fourth pattern activation duration
DD deactivation duration
DD1 first deactivation duration
DD1* first pattern deactivation duration
DD2 second deactivation duration
DD2* second pattern deactivation duration
DD3 third deactivation duration
DD3* third pattern deactivation duration
DE total duration of use of surgical instrument
DE* pattern total duration of use of surgical instrument
F fluid
f1 first frequency
f1* first pattern frequency
f2 second frequency
f2* second pattern frequency
f3 third frequency
f3* third pattern frequency
f4 fourth frequency
f4* fourth pattern frequency
IA electrical output current
INF information
K deviation criterion
M pattern parameter
P usage pattern
S control signal
ts starting point in time
te termination point in time
UA electrical output voltage
V type of use
X measure

1. A surgical system comprising:
a surgical instrument,
a surgical apparatus configured to connect to the surgical instrument and having a control unit configured to control at least one operation parameter (B) of the surgical instrument,
an evaluation unit communicatively connected to the surgical apparatus, wherein the evaluation unit is configured to identify a currently carried out or a planned type of use (V) of the surgical instrument based on at least one pattern parameter (M) for comparison to the at least one operation parameter (B) for determination and initiation of at least one measure (X, INF) to be carried out depending on the result of the comparison.

2. The surgical system according to claim 1, wherein the type of use (V) is characterized by a temporal sequence of settings of the at least one operation parameter (B).

3. The surgical system according to claim 1, wherein the at least one operation parameter (B) comprises an electrical signal provided to the surgical instrument and/or a fluid parameter of a fluid provided to the surgical instrument and/or an optical signal provided to the surgical instrument.

4. The surgical system according to claim 1, wherein the at least one operation parameter (B) can comprise one or more of the following operation parameters (B):
an amplitude (A1, A2, A3, A4) or a frequency (f1, f2, f3, f4) of an electrical output signal provided to the surgical instrument,
a crest factor of the electrical output signal,
a duty cycle of the electrical output signal if the electrical output signal is pulse width modulated,
a wave form or a mode of the electrical output signal,
a fluid pressure and/or a volume flow rate of a fluid (F) provided to the surgical instrument.

5. The surgical system according to claim 1, wherein the evaluation unit is configured to modify the operation parameter (B) or at least one of the operation parameters (B) as measure or at least one of the measures.

6. The surgical system according to claim 1, wherein the evaluation unit is further configured to determine a deviation (D) between the operation parameter (B) or at least one of the operation parameters (B) and the respectively assigned pattern parameter (M) as result or as one of the results of the comparison.

7. The surgical system according to claim 6, wherein the evaluation unit is further configured to determine and initiate the measure or one of the measures, if the deviation (D) fulfills a deviation criterion (K).

8. The surgical system according to claim 1, wherein the evaluation unit is configured to output information (INF) as measure or as at least one of the measures, wherein the information (INF) can comprise one or more of the following pieces of information:
warning about a deviation of the at least one operation parameter (B) from the at least one pattern parameter (M),
recommendation referring to a change of the operation parameter (B) or at least one of the operation parameters (B),
information referring to one or more steps to be carried out subsequently,
information referring to one or more required resources for one or more steps to be subsequently carried out,
information referring to a time duration for one or more steps to be subsequently carried out.

9. The surgical system according to claim 1, wherein the surgical apparatus is connected to a data management system by a communication network.

10. The surgical system according to claim 9, wherein the evaluation unit is configured to receive a piece of information from the data management system prior to the start of using the surgical instrument, which identifies the planned type of use (V).

11. The surgical system according to claim 9, wherein the surgical apparatus is configured to store the at least one of the operation parameters (B) during use of the surgical instrument or to transmit the at least one of the operation parameters (B) to the data management system.

12. The surgical system according to claim 1, wherein the evaluation unit is further configured to determine the at least one operation parameter (B) during use of the surgical instrument and to compare it with the at least one pattern parameter (M) of multiple usage patterns (P) and to identify the currently carried out type of use (V) in case of determination of a sufficient similarity between the at least one operation parameter (B) and the at least one pattern parameter (M).

13. The surgical system according to claim 12, wherein the evaluation unit is configured to determine an assessment of the use of the surgical instrument, which is carried out as result of the comparison.

14. A method of operating a surgical system, wherein the surgical system comprises a surgical apparatus and a surgical instrument connected thereto, wherein the surgical apparatus comprises a control unit configured to control at least one operation parameter (B) of the surgical instrument, wherein the method comprises:
identifying a currently carried out or planned type of use (V) of the surgical instrument,
assigning at least one pattern parameter (M) to the type of use (V),
comparing the at least one operation parameter (B) of the type of use (V) with the at least one pattern parameter (M) and
determining and initiating at least one measured (X, INF) to be carried out depending on a result of the comparison.
